SEQUENCE LISTING (2 sequences)

SEQ ID NO: 1 - DNA, 21 bp, Artificial Sequence
  PCR primer
tgacactggcaaaacaatgca

SEQ ID NO: 2 - DNA, 21 bp, Artificial Sequence
  PCR primer
ggtccttttcaccagcaagct

(19) United States
(12) Patent Application Publication
(10) Pub. No.: US 2014/0148482 A1
(43) Pub. Date: May 29, 2014

(54) METHOD FOR SCREENING ANTI-CANCER DRUGS AND METHOD OF CANCER TREATMENT

(71) Applicant: National Taiwan University, Taipei (TW)

(72) Inventors: Da-Liang Ou, Taipei City (TW); Chung-Wai Shiau, Changhua County (TW); Chiun Hsu, Taipei City (TW)

(73) Assignee: National Taiwan University, Taipei (TW)

(21) Appl. No.: 14/079,949

(22) Filed: Nov. 14, 2013

Related U.S. Application Data
(60) Provisional application No. 61/727,579, filed on Nov. 16, 2012.

(51) Int. Cl. G01N 33/50

(52) U.S. Cl. CPC G01N 33/5023
USPC 514/312; 514/597; 435/6.12; 514/347

(57) ABSTRACT

The present application provides a method for screening compounds for cancer treatment comprising treating cancer cells with a candidate compound for sufficient time for detectable expression of a gene selected from GADD45 family; and detecting the level of expression of the gene as compared to the level of expression in the absence of the candidate compound; wherein an increased level of expression in the presence of the candidate compound as compared to expression in the absence of the candidate compound indicates that the candidate compound acts as an inhibitor of the cancer cells. In some embodiments, the method can be used for screening compounds for treatment of sorafenib-resistant hepatocellular carcinoma. The present application further provides a method for treating hepatocellular carcinoma comprising administering to a subject having hepatocellular carcinoma a therapeutic effective amount of an agonist of GADD45 family.

### CROSS-REFERENCE TO RELATED APPLICATION(S)

This application claims priority to U.S. Provisional Patent Application No. 61/727,579, filed on Nov. 16, 2012, the disclosure of which is hereby incorporated by reference in its entirety.

### BACKGROUND OF THE INVENTION

1. Field of the Invention

The present invention relates to a method for screening compounds for cancer treatment, and more particularly to screen compounds for treatment of hepatocellular carcinoma.

2. Description of the Related Art

Cancer, also known as a malignant neoplasm, is a broad group of diseases involving unregulated cell growth. Typically, it is characterized by an increase in the number of abnormal cells derived from a normal tissue, invasion of adjacent tissues, and spread of the cancer cell to lymph nodes and distant sites via blood or lymphatic system. The neoplastic lesion may evolve clonally and develop an increasing capacity for invasion, growth, metastasis, and heterogeneity.

Hepatocellular carcinoma (HCC) is one of the most common tumors worldwide, which is the third leading cause of cancer deaths. Many patients with HCC remain asymptomatic until the disease is in its advanced stages, resulting in ineffective treatment and poor prognosis. Sorafenib, an oral multi-targeted kinase inhibitor targeting Raf kinase, vascular endothelial growth factor receptor (VEGFR)-2, VEGFR-3, and platelet-derived growth factor receptor, is currently the only drug approved for the treatment of advanced hepatocellular carcinoma (HCC) because of its survival benefits, which have been demonstrated by two randomized, placebo-controlled trials (Shen Y C et al. J Gastroenterol. 2010; 45:794-807).

Two important issues need to be addressed in order to facilitate future development of the drug. First, sorafenib was developed in a study that was designed to find specific Raf kinase inhibitors. However, preclinical models indicate that the antitumor activity of sorafenib does not correlate completely with its inhibitory effects on Raf/mitogen-activated protein kinase (MAPK), extracellular signal-regulated kinase (ERK), and kinase (MEK)/ERK activity (Wilhelm S et al. Nature reviews Drug discovery. 2006; 5:835-44). In addition, clinical trial results have indicated that the efficacy of sorafenib in HCC treatment might not be correlated with Raf/MEK/ERK signaling activity in HCC tumor samples (Abou-Alfa G K et al. J Clin Oncol. 2006; 24:4293-300; Newell P et al. J Hepatol. 2009; 51:725-33). Identification of the so-called “off-targeted effects” is critical for understanding the true anti-tumor mechanisms and for designing strategies to overcome drug resistance. In the past few years the present inventors have explored both the MEK/ERK-dependent and -independent mechanisms that could contribute to the antitumor activity of sorafenib in HCC (Ou D L et al. Cancer Res. 2010; 70:9309-18; Ou D L et al. Clin Cancer Res. 2009; 15:5820-8).

New drug development entails the identification of biomarkers that can predict clinical efficacy after molecular targeted therapy. The main effects of sorafenib in advanced HCC can be seen during tumor stabilization and survival prolongation. It is difficult to identify the sub-populations of patients who may benefit most from sorafenib treatment. It is also difficult to measure efficacy in individual patients because the objective response rate of sorafenib is quite low. Surrogate biomarkers to predict the biological and clinical efficacy of new drugs will help tailor treatment on an individual patient basis.

There is a need for a screening method for drugs for cancer treatment, especially for HCC treatment.

### SUMMARY

The present application provides a method for screening compounds for cancer treatment comprising: treating cancer cells with a candidate compound for sufficient time for detectable expression of a gene selected from GADD45 family; and detecting the level of expression of the gene as compared to the level of expression in the absence of the candidate compound; wherein an increased level of expression in the presence of the candidate compound as compared to expression in the absence of the candidate compound indicates that the candidate compound acts as an inhibitor of the cancer cells.

In other embodiments, the present application also provides a method for screening compounds for treatment of sorafenib-resistant hepatocellular carcinoma comprising: treating a sorafenib-resistant hepatocellular carcinoma cell line with a candidate compound for sufficient time for detectable expression of GADD45γ; and detecting the level of expression of GADD45γ as compared to the level of expression in the absence of the candidate compound; wherein an increased level of expression of GADD45γ in the presence of the candidate compound as compared to expression in the absence of the candidate compound indicates that the candidate compound acts as an inhibitor of the sorafenib-resistant hepatocellular carcinoma.

The present application further provides a method for treating hepatocellular carcinoma comprising administering to a subject having hepatocellular carcinoma a therapeutic effective amount of an agonist of GADD45 family.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 illustrates induction of GADD45β, GADD45γ and c-jun mRNA in Huh-7 cells.

FIG. 2 illustrates the viability of HCC cells and HUVEC after drug treatment.

FIG. 3 illustrates induction of GADD45β, GADD45γ and c-jun mRNA in (A) sorafenib-sensitive cell line and (B) sorafenib-resistant cell line.

FIG. 4A illustrates total-ERK, and phospho-ERK protein levels in HCC cells, and FIG. 4B illustrates GADD45β mRNA induction in Huh-7 cells.

FIG. 5A illustrates growth inhibition, and FIGS. 5B and 5C illustrate apoptosis induction of HCC cell lines with SC-20 treatment.

FIG. 6A-C illustrates in vivo evidence of the antitumor efficacy of SC-20.

FIG. 7A-C illustrates in vivo evidence of the antitumor efficacy of SC-20.

### DETAILED DESCRIPTION OF THE EMBODIMENTS

In the present application, the method for screening compounds for cancer treatment comprises treating cancer cells with a candidate compound for sufficient time for detectable expression of a gene selected from GADD45 family; and detecting the level of expression of the gene as compared to the level of expression in the absence of the candidate compound; wherein an increased level of expression in the presence of the candidate compound as compared to expression in the absence of the candidate compound indicates that the candidate compound acts as an inhibitor of the cancer cells.

In some embodiments, the cancer includes such as liver cancer, colorectal cancer, breast cancer, lung cancer, renal cancer, thyroid cancer, head and neck cancer, leukemia and the like. In one embodiment, the cancer is liver cancer, more preferably, the liver cancer is hepatocellular carcinoma (HCC).

The cancer cell used for the drug screening can be primarily cell or cell line. In one embodiment, the cancer cell can be liver cancer cell, for example, primarily cells obtained from the HCC tissues, or a HCC cell line.

In the present application, GADD45 family is applied as the biomarker for screening the anti-cancer drugs. The Growth Arrest and DNA Damage, or gadd45, genes, including GADD45α (originally termed gadd45), GADD45β (originally termed MyD118), and GADD45γ (originally termed CR6), are considered stress sensors that can modulate the response of mammalian cells to genotoxic/physiological stress, and that can modulate tumor formation. Gadd45 proteins are rapidly induced by genotoxic stress agents, as well as by terminal differentiation and apoptotic cytokines. GADD45 family proteins have been found to play important roles in cellular stress response, survival, senescence and apoptosis regulation, and are frequently underexpressed in various types of cancers, including HCC. Although GADD family proteins have been found to inhibit cell growth and to induce apoptosis in various cancer models (1, 11), they have also been shown to promote cell survival in various types of cells, including hematopoietic cells and hepatocytes (12-14). Although Gadd45 family members have much in common, they still differ in their regulation and function (15). This could be attributed to diversity in the spectrum of binding transcription factors. Until now, several regulatory elements have been found in the promoters of Gadd45 genes, as well as in Gadd45 family genes, in different chromosome locus (16, 17). In some preferred embodiments, the gene is GADD45γ, GADD45β or a combination thereof.

In some embodiments, the present application also provides a method for screening compounds for treatment of sorafenib-resistant hepatocellular carcinoma comprising: treating a sorafenib-resistant hepatocellular carcinoma cell line with a candidate compound for sufficient time for detectable expression of GADD45γ; and detecting the level of expression of GADD45γ as compared to the level of expression in the absence of the candidate compound; wherein an increased level of expression of GADD45γ in the presence of the candidate compound as compared to expression in the absence of the candidate compound indicates that the candidate compound acts as an inhibitor of the sorafenib-resistant hepatocellular carcinoma. In the present method, GADD45γ is the biomarker for drug screening. In some embodiments, GADD45β can be applied as the second biomarker.

In the present application, one or more additional gene(s) can be applied with the GADD45 family for screening drugs. In some embodiments, the additional gene includes, such as, c-jun.

In the present application, the expression of the gene such as GADD45γ is transcription level (e.g. RNA), translation level (e.g. polypeptide, protein) or a combination thereof. In one embodiment, the GADD45 protein can be a natural producing protein or a recombinant protein. In one embodiment, a reporter gene (e.g. the gene of a green fluorescent protein) fused with the biomarker gene or the promoter thereof can be used for detecting the gene expression.

The sufficient time for the candidate compound treatment can be determined based on the type of cancer, cell or cell line, dosage, formulation and/or other experimental parameters. In one embodiment, the treatment time can be 5 minutes, 15 minutes, 30 minutes, 1 hour, 6 hours, 12 hours, 18 hours, 1 day, 2 days, 3 days or the like.

The target cancer cell without treatment of the candidate compound is used as control group, and the expression of said gene in the control group is determined as basal line. The test group, i.e. the target cancer cell treated with the candidate compound, is compared with the control group to determine the change of the gene expression affected by the candidate compound. Optionally, the target cancer cell treated with the conventional drug can be used as positive control group, and, preferably, the conventional drug is known as anti-cancer drug with well-established therapeutic effects. The positive control group can be a reference to further evaluate the therapeutic effects of the candidate compound.

In one embodiment, the expression of GADD45γ in the absence of the candidate compound (i.e. without treatment) is determined as 100%. While the expression of GADD45γ in the presence of the candidate compound is 300% or above, it can be determined that the candidate compound increases the expression level of GADD45γ and has potential to be an agonist of GADD45γ as well as an anti-cancer drug. In one embodiment, the expression of GADD45γ in the presence of the candidate compound can be more than 350%, 400%, 500%, 800%, 1000%, 1200%, or even a higher value.

In another embodiment, it can be determined that the candidate compound increases the expression level of GADD45β and has potential to be an agonist of GADD45β as well as an anti-cancer drug while the expression of GADD45β in the presence of the candidate compound is 700% or above, for example, 750%, 800%, 1000%, 1200%, 1600%, 2200%, 2500%, or even a higher value.

In another embodiment, while c-jun is applied as a biomarker, its increased level of expression is preferably more than 250%, for example, 300%, 350%, 400%, 500%, 600%, or even a higher value.

The present application further provides a method for treating hepatocellular carcinoma comprising administering to a subject having hepatocellular carcinoma a therapeutic effective amount of an agonist of GADD45 family. In a preferred embodiment, the agonist enhances the expression of GADD45γ, GADD45β or both in the treated subject. In a preferred embodiment, the present method can be applied to treat a subject having sorafenib-resistant hepatocellular carcinoma.

In the present application, the subject refers to a vertebrate, preferably a mammal, more preferably a human. Mammals include, but are not limited to, murines, simians, humans, pets, farm animals, and the like. In some embodiments, the subject can be human, monkey, primates, cat, dog, rat, mouse, porcine, horse, goat, sheep, cattle and the like.

The therapeutic effective amount of an agonist of GADD45 family can be determined by methods well-known in the art. In some embodiments, the effective amount can be about 1-1000 mg per day, such as 1-500 mg, 1-250 mg, 1-100 mg, 5-500 mg, 5-250 mg, 5-100 mg, and the like.

In some embodiments, the agonist can be selected from a group consisting of:

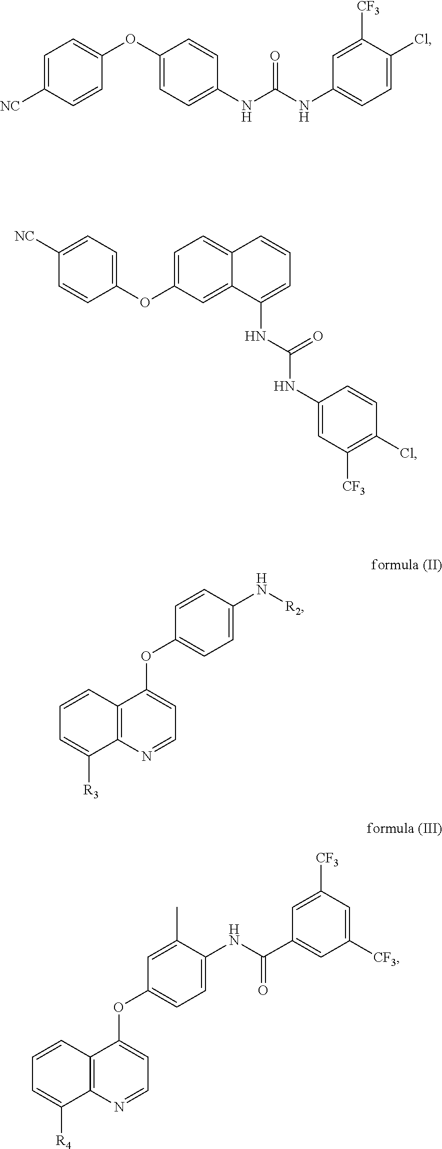

and a pharmaceutically acceptable salt, solvate or stereoisomer thereof;
wherein R₂ is

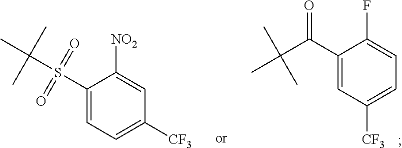 [SMILES: CC(C)(C)S(=O)(=O)c1ccc(C(F)(F)F)cc1[N+](=O)[O-] ; "or" ; CC(C)(C)C(=O)c1cc(C(F)(F)F)ccc1F ; ";"]

### R₃ is H; and R₄ is NO₂.

### EXAMPLES

The inventors found that GADD45β and GADD45γ, which are associated with cellular stress response and apoptosis regulation, were induced in HCC cells after the sorafenib treatment. GADD45β and GADD45γ inductions were more prominent in sorafenib-sensitive HCC cells than in sorafenib-resistant HCC cells, and were independent of any MEK/ERK signaling. It was also found that JNK/c-Jun signaling induction was critical for the induction of GADD45β, and that the inhibition of JNK/c-Jun signaling could partially antagonize any sorafenib-induced GADD45β expression or apoptosis in HCC cells. These findings support the roles of GADD45β and GADD45γ as predictive biomarkers of sorafenib efficacy in HCC.

Therefore, the inventors hypothesize that the modulation of GADD45β and GADD45γ and c-Jun signaling pathways may reverse sorafenib resistance and serve as new therapeutic targets in HCC.

Roles of GADD45 Family Proteins and JNK/c-Jun Signaling in HCC

GADD45 family proteins, including GADD45α, GADD45β, and GADD45γ, have been found to play important roles in cellular stress response, survival, senescence and apoptosis regulation, and are frequently underexpressed in various types of cancers, including HCC.

JNK/c-Jun signaling may be an important contributor to the various effects of GADD45 family proteins on different cellular contexts. All three GADD45 proteins can interact directly with, and activate, p38 kinase, as well as indirectly activate a JNK cascade in order to induce programmed cell death. Ectopic expressions of all three GADD45 proteins were shown to induce apoptosis in HeLa cells and to enhance stress-mediated apoptosis in both M1 leukemia and H1299 lung carcinoma cells. On the other hand, GADD45α and GADD45β can protect cells from genotoxic stress using two distinct mechanisms: the activation of a p38-NF-κB-mediated survival pathway, and the inhibition of the stress response MKK4-JNK pathway, respectively. These findings showed that the JNK pathway seems to have a dual role in cellular survival, depending on the cell types and the types of stress. JNK1 activation has been found to be an important factor in promoting hepatocarcinogensis and may serve as a poor prognostic marker for HCC. In chemical-induced mouse HCC models, JNK1 was activated and was associated with dysregulated cell cycle control as well as with increased proliferation. Increased JNK1 expression in human HCC tumor tissue was associated with advanced tumor stages and poor prognosis. The gene expression profiles of these JNK1-overexpressed tumors revealed that the up-regulated genes in these JNK1-overexpressed tumors are mostly associated with a hepatic progenitor cell phenotypes. On the other hand, JNK1 signaling is an essential mediator for transforming growth factor-beta- and adiponectin-induced apoptosis in HCC cells. The above data illustrates the dual functional roles (oncogenic and tumor-suppressive) that JNK signaling may play in HCC. In addition, JNK signaling is also important in mediating the cytotoxic effects induced by chemotherapeutic agents. The above data illustrates the complexity of exploring GADD45 and JNK signaling pathways as therapeutic targets or as predictive biomarkers in HCC.

Novel Compounds that can Overcome Sorafenib Resistance Through the Regulation of GADD45β and GADD45γ Signaling

The present inventors tested the novel candidate compounds in HCC cells and found that some of these compounds, e.g. SC-20 and SC-72, showed an impressive induction of GADD45, as well as an inhibition of cell growth. From the lead compounds, a structure activity relationship based on the SC-20 or the SC-72 skeleton was established. Therefore, it is planned to design an efficient synthetic route for generating a series of SC-20 derivatives and a series of SC-72 derivatives. The detailed routes are shown below.

SC-20:

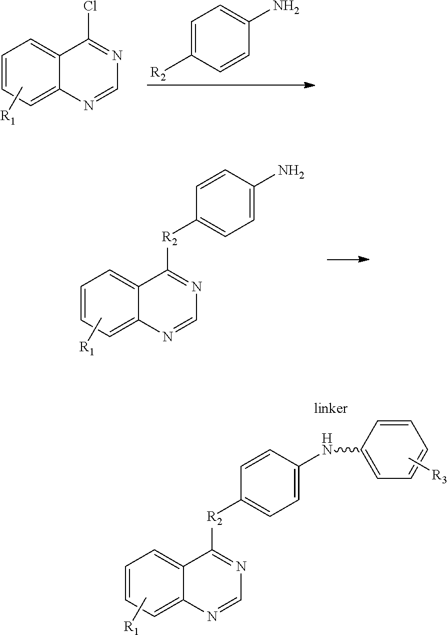

There are two steps and three components that make up the synthetic route. Each of these three components can be replaced by similar isosteric chemicals. For example, the quinoline could be replaced with different heterocyclic rings, such as quinazoline. The phenyl ring of the SC-20 structure could be replaced with naphthalene or a connected phenyl ring with a functional group.

SC-72:

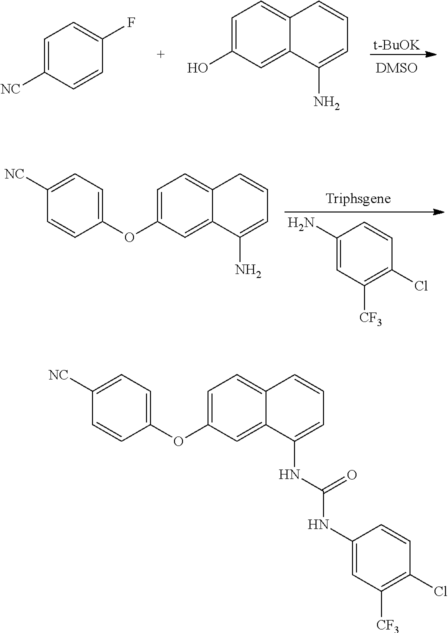

The structures of the series of SC-20 and SC-72 derivatives are shown as follows.

Formula (I)

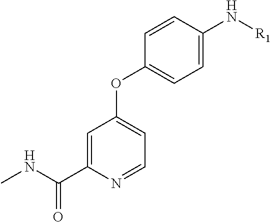

| | R₁ |
|---|---|
| SC-1 | 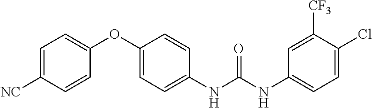 |
| SC-2 | 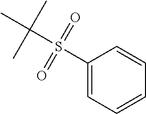 |
| SC-3 | 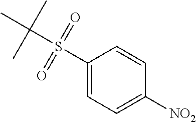 |
| SC-4 | 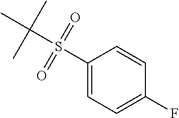 |
| SC-5 | 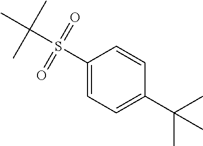 |
| SC-6 | 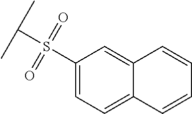 |
| SC-7 | 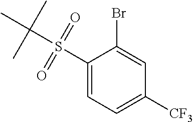 |
| SC-8 | 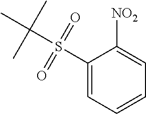 |
| SC-9 | 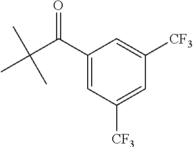 |
| SC-10 | 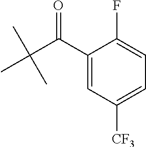 |
| SC-11 | 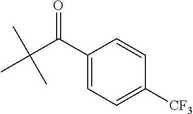 |

Formula (II)

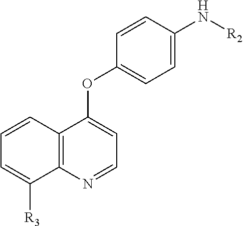

| | R₂ | R₃ |
|---|---|---|
| SC-12 | H | 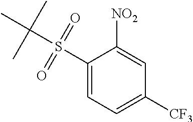 |
| SC-13 | NO₂ | 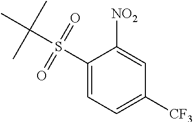 |
| SC-14 | H | 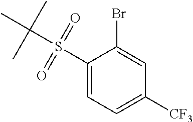 |
| SC-15 | NO₂ | 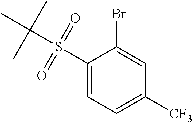 |
| SC-16 | H | 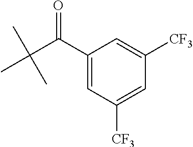 |
| SC-17 | NO₂ | 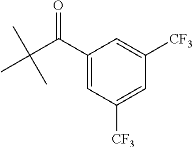 |
| SC-18 | H | 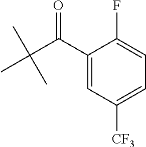 |
| SC-19 | NO₂ | 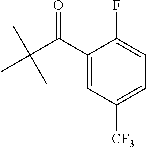 |

Formula (III)

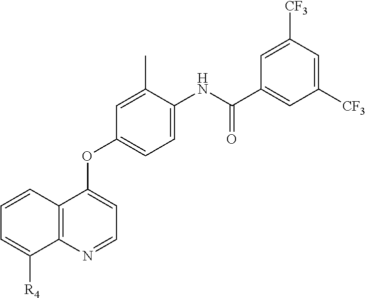

| | R₄ |
|---|---|
| SC-20 | NO₂ |
| SC-21 | NH₂ |
| SC-22 | NHCOCH₃ |
| SC-72 | 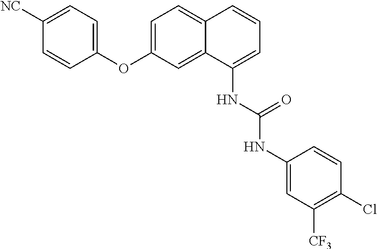 |

### Example 1

### Drugs Screening Platform

It was tested a series of the above derivatives lacking Raf inhibitor activity for their effects on GADD45 induction in HCC cells.

For in vitro experiments, the candidate compounds (e.g. the series of SC-20 and SC-72 derivatives) were dissolved in DMSO, and the final concentration of DMSO was kept below 0.1%. For in vivo experiments, SC-20 and sorafenib was dissolved in Cremophor EL/95% ethanol (50:50; Sigma-Aldrich).

Cell Culture and Candidate Compound Treatment

Huh-7 cells, a HCC cell line, were cultured in Dulbecco's modified Eagle's medium (DMEM), containing 10% fetal bovine serum, penicillin (100 units/mL), streptomycin (100 μg/mL), L-glutamine (2 mmol/L), and sodium pyruvate (1 mmol/L) at 37° C. in a humidified incubator containing 5% CO2.

Huh-7 cells, were added with 10 μM of the candidate compound and co-cultured under the same conditions for 24 hours. Sorafenib (10 μM) is used as a positive control group, and Huh-7 cells without compound treatment is used as the reference control group. mRNA levels were assessed through quantitative RT-PCR.

Quantitative Reverse Transcriptase Polymerase Chain Reaction (qRT-PCR)

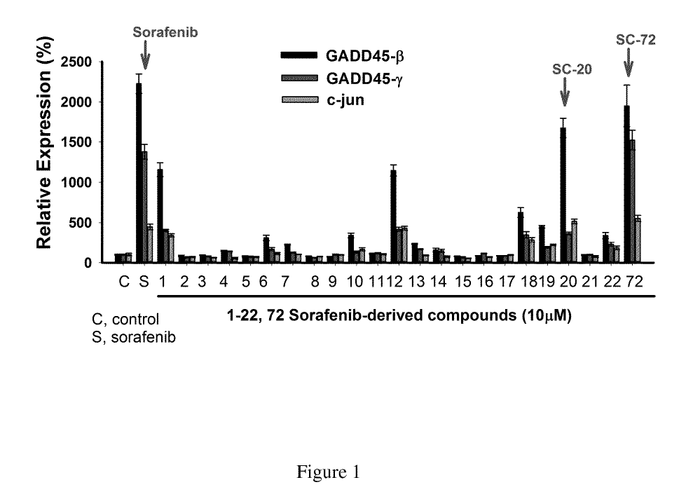

RNA was extracted using a Trizol reagent (Invitrogen, San Diego, Calif.). cDNAs were synthesized from total RNA (1 μg) using a High Capacity cDNA Archive kit (Applied Biosystems, Foster City, Calif.) and quantified using the TaqMan-Universal or SYBR Green PCR Master Mix (Applied Biosystems, Foster City, Calif.) on an ABI PRISM 7900 Sequence Detection System (Applied Biosystems, Foster City, Calif.). The primers for the GADD45β, GADD45γ and c-jun genes were purchased from Applied Biosystems (ABI TaqMan assay ID: Hs00169587_ml, Hs00198672_ml and Hs00277190_s1). Primers for the hypoxanthine phosphoribosyltransferase (HPRT; sense 5′-TGACACTGGCAAAACAATGCA-3′ and antisense 5′-GGTCCTTTTCACCAGCAAGCT-3′) gene were used as endogenous controls. Conditions for PCR were as follows: 50° C. for 2 minutes, 95° C. for 10 minutes, and then 40 cycles of 95° C. for 15 seconds (denaturation) and 60° C. for 1 minute (annealing/extension). The relative mRNA amount of the target/control genes was calculated using the ΔCt (threshold cycle) method: relative expression=2−ΔCt, where ΔCt=Ct (target gene)−Ct (control gene). The gene expression of the reference control group is as 100%. The experiment results were shown in FIG. 1 and Table 1.

FIG. 1 shows induction of GADD45β, GADD45γ and c-jun mRNA in Huh-7 cells after treatments. Several derivatives have the same patterns of GADD45 induction as sorafenib does. The values of the relative gene expressions are shown as Table 1.

TABLE 1

| | GADD45β | | c-Jun | | GADD45γ | |
|---|---|---|---|---|---|---|
| | Mean | sd | Mean | sd | Mean | sd |
| reference | 100.00 | 7.12 | 100.00 | 15.91 | 100.00 | 7.73 |
| control group | | | | | | |
| sorafenib | 2226.40 | 120.58 | 447.28 | 35.26 | 1377.54 | 92.58 |
| SC-1 | 1156.44 | 85.88 | 343.08 | 20.87 | 402.46 | 14.30 |
| SC-2 | 85.09 | 9.06 | 69.51 | 7.49 | 67.49 | 7.24 |
| SC-3 | 91.85 | 4.63 | 62.91 | 3.42 | 77.62 | 5.74 |
| SC-4 | 146.97 | 7.60 | 57.29 | 10.03 | 139.93 | 4.97 |
| SC-5 | 78.30 | 8.43 | 69.16 | 7.58 | 72.74 | 9.69 |
| SC-6 | 310.68 | 34.75 | 112.47 | 12.89 | 167.06 | 20.78 |
| SC-7 | 223.29 | 7.04 | 102.50 | 1.29 | 124.59 | 7.92 |
| SC-8 | 80.34 | 3.86 | 74.76 | 2.92 | 62.03 | 4.02 |
| SC-9 | 73.85 | 1.77 | 94.95 | 4.80 | 98.49 | 8.52 |
| SC-10 | 342.04 | 29.14 | 164.98 | 20.32 | 132.14 | 12.88 |
| SC-11 | 108.50 | 7.47 | 104.64 | 7.15 | 116.60 | 8.09 |
| 5C-12 | 1145.95 | 69.45 | 431.22 | 24.20 | 418.30 | 23.56 |
| 5C-13 | 231.24 | 6.79 | 89.56 | 5.86 | 167.11 | 6.20 |
| 5C-14 | 157.99 | 21.40 | 73.17 | 9.73 | 145.58 | 20.01 |
| SC-15 | 77.93 | 4.98 | 55.24 | 3.55 | 65.59 | 7.96 |
| SC-16 | 83.52 | 4.86 | 69.38 | 4.88 | 110.20 | 6.44 |
| 5C-17 | 85.81 | 3.76 | 93.91 | 5.58 | 86.10 | 2.55 |
| SC-18 | 625.80 | 60.18 | 285.75 | 29.08 | 348.22 | 38.90 |
| SC-19 | 449.32 | 15.82 | 220.07 | 9.45 | 190.55 | 10.26 |
| SC-20 | 1674.94 | 122.40 | 515.47 | 29.17 | 364.16 | 17.20 |
| SC-21 | 94.65 | 5.71 | 77.51 | 10.08 | 96.65 | 6.60 |
| SC-22 | 340.21 | 37.90 | 183.54 | 23.35 | 229.94 | 26.16 |
| SC-72 | 1951.02 | 257.45 | 552.00 | 37.75 | 1522.00 | 122.00 |

While a candidate compound resulted in 300% or above of the GADD45γ expression, 800% or above of the GADD45β expression, or both, said candidate compound can be determined as a potential anti-cancer drug and can be selected for further study and evaluation. As shown in Table 1, SC-1, SC-12, SC-18, SC-20 and SC-72 satisfied the increased expression of GADD45 family. SC-20 and SC-72 were selected for further experiments.

### Example 2

### In Vitro Evidence of the Candidate Compounds

### Cell viability was assessed using an MTT (3-(4,5-dimethylthiazol-2-yl)-2,5-diphenyltetrazolium bromide) assay

The HCC cell line, Hep3B, was obtained from the American Type Culture Collection (ATCC), and the Huh-7 cell line was from the Health Science Research Resources Bank. Cells were cultured in DMEM, containing 10% fetal bovine serum, penicillin (100 units/mL), streptomycin (100 μg/mL), L-glutamine (2 mmol/L), and sodium pyruvate (1 mmol/L) at 37° C. in a humidified incubator containing 5% CO2. A sorafenib-resistant cell line, Huh-7R, was generated through continuous treatment of Huh-7 cells with sorafenib up to 10 μmol/L.Human umbilical vein endothelial cells (HUVEC) were procured from ScienCell Research Laboratories, CA. HUVECs were maintained in endothelial cell culture medium (ScienCell) to selectively promote growth at 37° C. in a 5% CO₂ incubator.

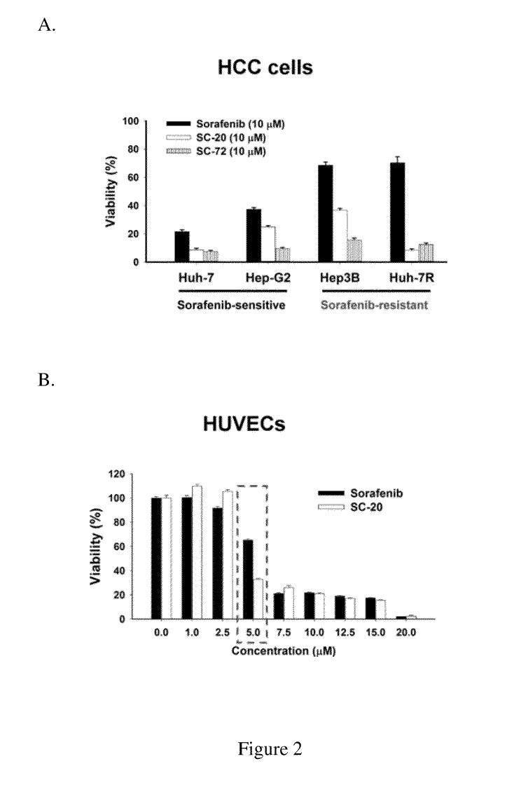

HCC cells and HUVEC (human umbilical venous endothelial cells) were treated with the candidate compound (SC-20 or SC-72) for 72 hours. The compound concentrations and the conditions of treatment are same as Example 1. Then the cells were collected and measured via an MTT assay. The results were shown in FIGS. 2A and 2B, the bars indicate the percentage of surviving cells after 72 hours of drug treatment.

In FIG. 2A, SC-20 and SC-72 appeared even more potent than sorafenib for the growth inhibition of HCC cells, especially in sorafenib-resistant cell lines (Hep3B and Huh-7R). For example, the decreased cell viability of Hep3B by sorafenib, SC-20 and SC-72 were, 68.57, 36.78 and 15.65 respectively. In FIG. 2B, SC-20 also appeared even more potent than sorafenib for the growth inhibition of HUVECs, especially in 5 uM compounds treatments.

### Example 3

### GADD45γ, more than GADD45β, as a Screening Biomarker

SC-20 was then tested for GADD45β, GADD45γ and c-Jun mRNA in sorafenib-sensitive (Huh-7) and sorafenib-resistant (Huh-7R) cell lines. The cell culture and the steps of SC-20 treatment were as described above. The concentration of SC-20 and was 10 μm. Said mRNA was measured via qRT-PCR.

qRT-PCR:

RNA was extracted using a Trizol reagent (Invitrogen, San Diego, Calif.). cDNAs were synthesized from total RNA (1 μg) using a High Capacity cDNA Archive kit (Applied Biosystems, Foster City, Calif.) and quantified using the TaqMan-Universal or SYBR Green PCR Master Mix (Applied Biosystems, Foster City, Calif.) on an ABI PRISM 7900 Sequence Detection System (Applied Biosystems, Foster City, Calif.). The primers for the GADD45β, GADD45γ and c-jun genes were purchased from Applied Biosystems (ABI TaqMan assay ID: Hs00169587_ml, Hs00198672_ml and Hs00277190_sl). Primers for the hypoxanthine phosphoribosyltransferase (HPRT; sense 5′-TGACACTGGCAAAACAATGCA-3′ and antisense 5′-GGTCCTTTTCACCAGCAAGCT-3′) gene were used as endogenous controls. Conditions for PCR were as follows: 50° C. for 2 minutes, 95° C. for 10 minutes, and then 40 cycles of 95° C. for 15 seconds (denaturation) and 60° C. for 1 minute (annealing/extension). The relative mRNA amount of the target/control genes was calculated using the ΔCt (threshold cycle) method: relative expression=2−ΔCt, where ΔCt=Ct (target gene)−Ct (control gene).

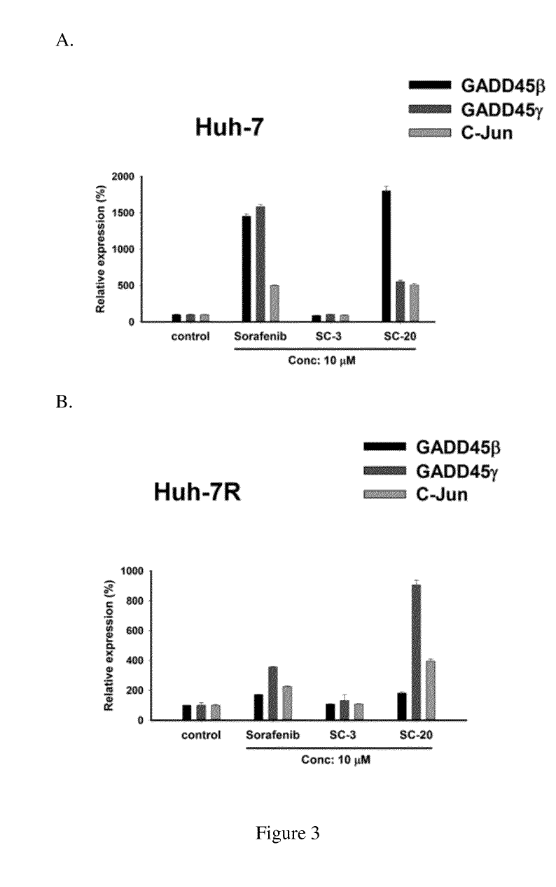

The present examples set the cut-off at more than 300%. It was found that GADD45γ can be induced by SC-20, both in sorafenib-sensitive (Huh-7) and sorafenib-resistant (Huh-7R) cell lines (FIGS. 3A and 3B). The results suggest that GADD45γ may be a more accurate predictive biomarker than GADD45β, when the drug is effective in sorafenib-resistant (Huh-7R) cell lines.

### Example 4

### Antitumor Efficacy of SC-20

### Example 4.1

### SC-20 Lacks Raf/MEK/ERK Inhibitor Activity

Previous studies have suggested that sorafenib is inhibition of Raf kinase. However, induction of GADD45β effect is unrelated to RAF kinase inhibition. This example is to determine whether SC-20 involves the pathway as sorafenib does.

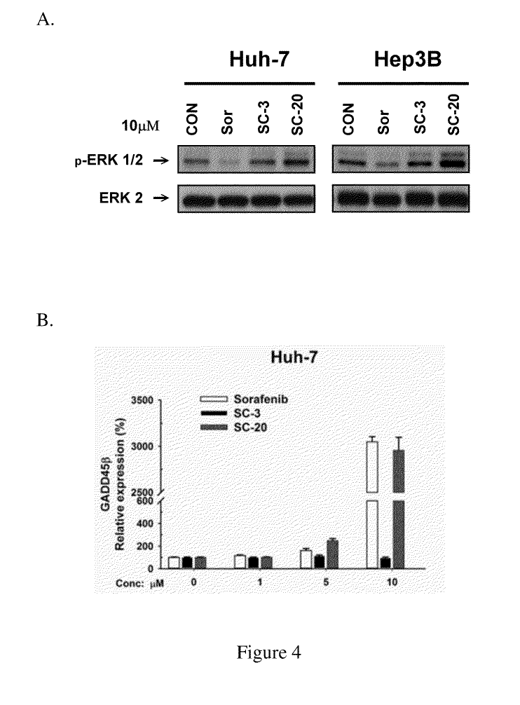

Total-ERK, and phospho-ERK protein levels in HCC cells after sorafenib, SC-3 or SC-20 treatment at 24 hours. Whole-cell lysates after drug treatment were examined using Western blotting. Whole cell lysates of HCC cells after drug treatment were prepared and quantified as previously described (Ou D L et al. Cancer Res. 2010; 70:9309-18). SDS-PAGE and Western blot analysis were performed to measure protein expression. Signals were visualized using a UVP Imaging System (UVP, Upland, Calif.), or with X-ray film. The result was shown in FIG. 4A. It was found that SC-20, not the Raf kinase inhibitor, is incapable of reducing p-ERK in Huh-7 and Hep3B cells.

The effects of GADD45β mRNA induction by different concentrations of the candidate compounds were tested. Huh-7 cells were treated with sorafenib, SC-3 or SC-20 for 24 hours, and GADD45β mRNA levels were assessed using real-time qRT-PCR. The result was shown in FIG. 4B. It was found that 10 uM sorafenib and SC-20 significantly induced the GADD45β mRNA expression.

### Example 4.2

### In Vitro Evidence of the Antitumor Efficacy of SC-20

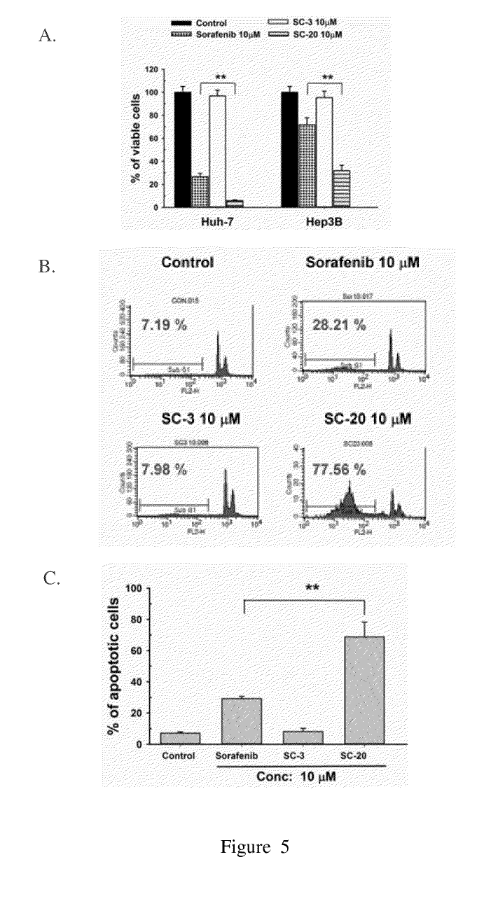

Huh-7 and Hep3B cells were treated with sorafenib, SC-3 or SC-20 for 72 hours, and the cell viability was assessed using an MTT assay. The result was shown in FIG. 5A. **, P<0.01, compared with sorafenib group.

The treated Huh-7 cells Sub-G1 was assessed using flow cytometry, and the result was shown in FIGS. 5B and 5C. FIG. 5C shows triplicate results through independent experiments. **, P<0.05, compared with the sorafenib group.

FIG. 5A results of the in vitro antitumor efficacy, SC-20 displayed more potency than sorafenib, both in sorafenib-sensitive (Huh-7) or sorafenib-resistant (Hep3B) HCC cell lines, significantly inhibited cell growth. In addition, SC-20 displayed more potency than sorafenib in inducing apoptosis (FIGS. 5B and C) in Huh-7.

### Example 4.3

### Mice and Subcutaneous Xenograft Model of HCC

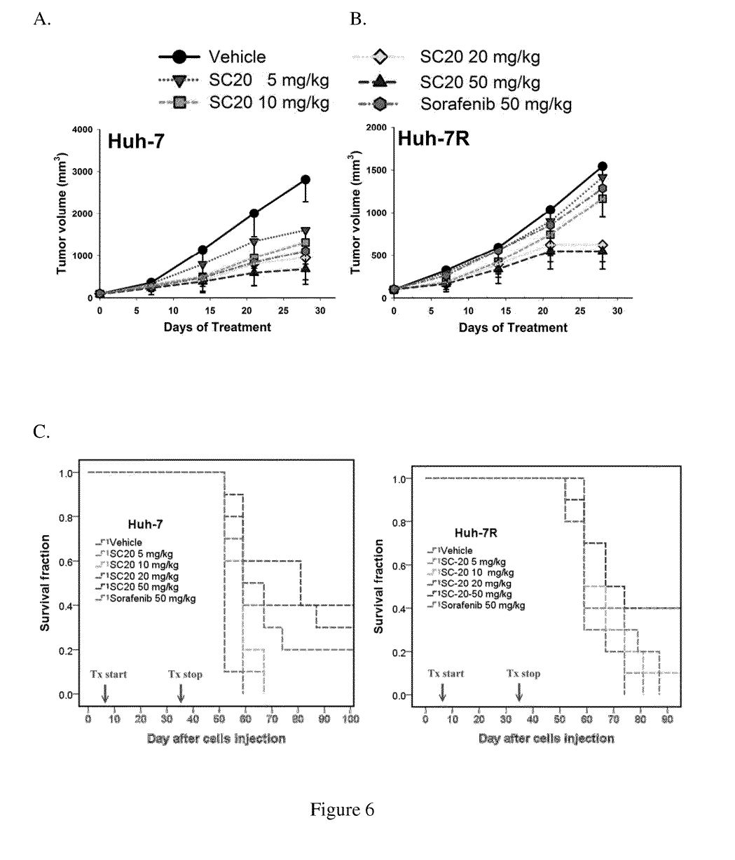

The protocol for the in vivo studies was approved by the Institutional Animal Care and Use Committee of College of Medicine, National Taiwan University. All the animal studies were performed according to the criteria outlined in the Guide for the Care and Use of Laboratory Animals prepared by National Academy of Sciences and published by NIH. Male BALB/c athymic (nu+/nu+) mice were inoculated subcutaneously with Huh-7 cells or Huh-7R cells (˜1×10⁶). When the tumor volume reached ˜100 mm3 [volume (mm3)=(width)2×length×0.5], the mice were randomized into six treatment groups (n≧6 in each group): (a) vehicle control, (b) SC-20: 5 mg/kg/d, (c) SC-20: 10 mg/kg/d, (d) SC-20: 20 mg/kg/d, (e) SC-20: 50 mg/kg/d and (f) sorafenib: 50 mg/kg/d. Drug treatment was given daily via gavage. Tumor volume and body weight were recorded every 7 days. Tumor images from mice are from sacrifices at 25 days of treatment. The results were shown in FIGS. 6A and 6B.

An additional mice were used to establish xenografts to observe survival time. For these experiments, mice with subcutaneously tumors (100 mm³) were treated as described above. Survival was monitored until the experiments were terminated due to heavy tumor burden over 2000 mm³ (FIG. 6C). Tx start, treatment start time; Tx stop, treatment stop time.

In the preliminary results of the in vivo anti-cancer effect it can be seen that SC-20 (20 mg/kg/d) significantly inhibited tumor growth more than highly dose of sorafenib (50 mg/kg/d) in a sorafenib-resistant (Huh-7R) xenograft model (FIG. 6B). In addition, SC-20 (20 mg/kg/d) were significantly inhibited tumor growth more than highly dose of sorafenib (50 mg/kg/d) in a sorafenib-sensitive (Huh-7) xenograft model (FIG. 6A). In addition, based on animal survival studies showed SC-20 (20 mg/kg/d) significantly prolong mice survival times more than highly dose of sorafenib (50 mg/kg/d) both in sorafenib-sensitive (Huh-7) and in sorafenib-resistant (Huh-7R) xenograft model (FIG. 6C).

### Example 4.4

### Mice and the Orthotopic Allograft Model of HCC

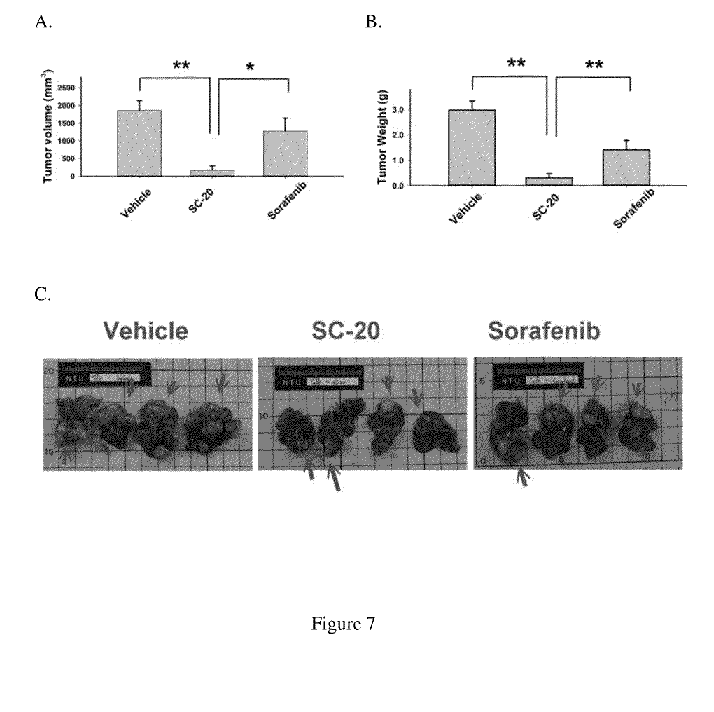

The protocol for the in vivo studies was approved by the Institutional Animal Care and Use Committee of College of Medicine, National Taiwan University. All the animal studies were performed according to the criteria outlined in the Guide for the Care and Use of Laboratory Animals prepared by National Academy of Sciences and published by NIH. BALB/c mice were anaesthetized with isoflurane, and a small transverse incision was made in the left abdomen. BNL-MEA cells (˜1.5×10⁵) suspended in 20 μl PBS were slowly injected with a 30-gauge needle into the left liver lobe, which was exposed through the incision. After injection, the incision was closed by suture with absorbable material. Five days after the surgery, the mice were randomized to three treatment groups (n≧4 in each group): (a) vehicle control, (b) SC-20 20 mg/kg/d and (c) sorafenib 10 mg/kg/d. Drug treatment was given daily via gavage. Mice sacrificed after twenty-five treatments were used as end-points of the therapeutic effect. The results were shown in FIGS. 7A, 7B and 7C. In FIG. 7C, the arrows indicated the tumors.

In the preliminary results of the in vivo anti-cancer effect, SC-20 in a BNL-MEA Allograft mice model significantly inhibited tumor growth in terms of tumor volume and weight (FIGS. 7A and 7B). In addition, treatments with SC-20 inhibited tumor growth significantly more than sorafenib in the Orthotopic model (FIG. 7C).

While the present invention is disclosed by reference to the preferred embodiments and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims and its equivalent systems and methods.

What is claimed is:

1. A method for screening compounds for cancer treatment comprising:
treating cancer cells with a candidate compound for sufficient time for detectable expression of a gene selected from GADD45 family; and
detecting the level of expression of the gene as compared to the level of expression in the absence of the candidate compound;
wherein an increased level of expression in the presence of the candidate compound as compared to expression in the absence of the candidate compound indicates that the candidate compound acts as an inhibitor of the cancer cells.

2. The method of claim 1, wherein the cancer is selected from the group consisting of liver cancer, colorectal cancer, breast cancer, lung cancer, renal cancer, thyroid cancer, head and neck cancer, and leukemia.

3. The method of claim 2, wherein the liver cancer is hepatocellular carcinoma.

4. The method of claim 1, wherein the cancer cell is a hepatocellular carcinoma cell line.

5. The method of claim 1, wherein the gene is GADD45γ, GADD45β or a combination thereof.

6. The method of claim 1, wherein the gene further comprises c-jun.

7. The method of claim 1, wherein the expression of the gene is transcription level, translation level or a combination thereof.

8. The method of claim 1, wherein the increased level of expression in the presence of the candidate compound is 300% or above while the expression in the absence of the candidate compound is determined as 100%.

9. The method of claim 1, wherein the increased level of expression in the presence of the candidate compound is 700% or above while the expression in the absence of the candidate compound is determined as 100%.

10. A method for screening compounds for treatment of sorafenib-resistant hepatocellular carcinoma comprising:
treating a sorafenib-resistant hepatocellular carcinoma cell line with a candidate compound for sufficient time for detectable expression of GADD45γ; and
detecting the level of expression of GADD45γ as compared to the level of expression in the absence of the candidate compound;
wherein an increased level of expression of GADD45γ in the presence of the candidate compound as compared to expression in the absence of the candidate compound indicates that the candidate compound acts as an inhibitor of the sorafenib-resistant hepatocellular carcinoma.

11. The method of claim 10, wherein the gene further comprises GADD45β, c-jun, or a combination thereof.

12. The method of claim 10, wherein the expression of GADD45γ is transcription level, translation level or a combination thereof.

13. The method of claim 10, wherein the increased level of expression of GADD45γ in the presence of the candidate compound is 300% or above while the expression in the absence of the candidate compound is determined as 100%.

14. A method for treating hepatocellular carcinoma comprising administering to a subject having hepatocellular carcinoma a therapeutic effective amount of an agonist of GADD45 family.

15. The method of claim 14, wherein the subject is mammalian.

16. The method of claim 14, wherein the agonist enhances the expression of GADD45γ, GADD45β or a combination thereof.

17. The method of claim 14, wherein the hepatocellular carcinoma comprises sorafenib-resistant hepatocellular carcinoma.

18. The method of claim 14, wherein the agonist is selected from a group consisting of: 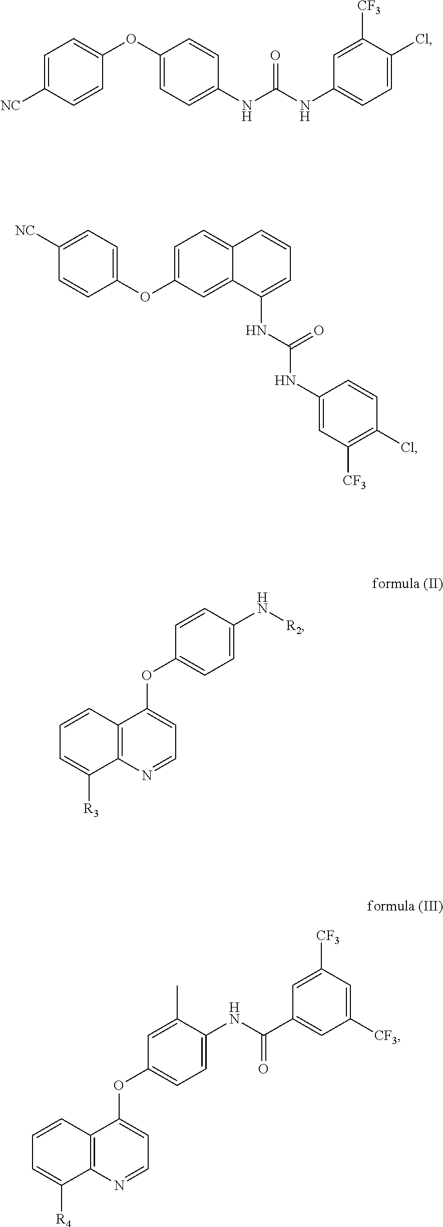
and a pharmaceutically acceptable salt, solvate or stereoisomer thereof;
wherein R₂ is 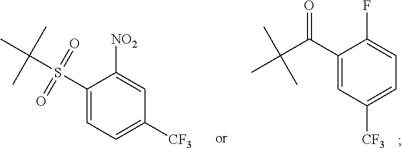 [SMILES: CC(C)(C)S(=O)(=O)c1ccc(C(F)(F)F)cc1[N+](=O)[O-] ; "or" ; CC(C)(C)C(=O)c1cc(C(F)(F)F)ccc1F ; ";"]
R₃ is H; and R₄ is NO₂.
